(19) United States
(12) Patent Application Publication
(10) Pub. No.: US 2020/0245929 A1
(43) Pub. Date: Aug. 6, 2020

(54) LIPID MEASURING APPARATUS AND LIPID MEASURING METHOD

(71) Applicant: MEDICAL PHOTONICS CO., LTD., Hokkaido (JP)

(72) Inventors: Kazuya IINAGA, Sapporo-shi, Hokkaido (JP); Takashi TSURUMI, Ebina-shi, Kanagawa (JP)

(21) Appl. No.: 16/482,075

(22) PCT Filed: Jan. 29, 2018
(86) PCT No.: PCT/JP2018/002679

§ 371 Date: Jul. 30, 2019

(30) Foreign Application Priority Data
Jan. 31, 2017 (JP) 2017-016012

(51) Int. Cl. A61B 5/00; G01N 21/47

(52) U.S. Cl. CPC A61B 5/4872; A61B 5/4866; A61B 5/74; A61B 5/0059; G01N 21/4795

(57) ABSTRACT

An apparatus and a method that readily allow noninvasive lipid measurement with no skill of a measurer. The apparatus includes an irradiator that radiates light having a predetermined light intensity to a predetermined site of a living body from outside the living body toward inside the living body, a light intensity detector that detects a light arrival range in the living body based on the light intensity of light emitted from the living body, and a controller that calculates a predetermined light arrival range parameter based on the light arrival range and calculates lipid concentration in the living body based on the light arrival range parameter.

### TECHNICAL FIELD

The present invention relates to a lipid measuring apparatus and a lipid measuring method.

### BACKGROUND ART

Attention has been directed to postprandial hyperlipidemia as a risk factor for arteriosclerosis. There has been a report stating that an increase in the concentration of neutral lipid in a non-hunger state increases the risk of development of an event of coronary artery diseases.

To diagnose postprandial hyperlipidemia, it is necessary to observe a change in in-blood lipid concentration for 6 to 8 hours after meals. That is, to measure the state of hyperlipemia, it is necessary to place a subject under restraint for 6 to 8 hours and collect blood multiple times. The diagnosis of postprandial hyperlipidemia is therefore no better than clinical studies, and diagnosing postprandial hyperlipidemia at a clinical site is not practical.

Patent Literature 1 discloses an approach to a solution of the problem described above. According to the approach disclosed in Patent Literature 1, noninvasive lipid measurement can eliminate blood collection. The in-blood lipid can therefore be measured not only in a medical institution but at home. Allowing instantaneous data acquisition allows temporally continuous in-blood lipid measurement.

### CITATION LIST

### Patent Literature

Patent Literature 1

International Publication No. 2014/087825

### SUMMARY OF INVENTION

### Technical Problem

In the noninvasive lipid measuring approach shown in Patent Literature 1, however, determination of an optimum measurement site requires skill of a measurer, causing a measurement error.

When light passes through a living body, the skin, muscle, blood, and other factors attenuate the intensity of the light. To detect the concentration of a specific substance in a living body, it is desirable to minimize influences other than a target under measurement.

On the other hand, since the precision of measurement is expressed by the ratio of a signal to noise (S/N), it can be said that the measurement precision can be improved by detection of an increased intensity of a signal from the target under measurement.

The measurement approach shown in Patent Literature 1, although it is based on one-dimensional (linear) detection, has a difficulty in measurement at a single site due to positional displacement of a measurement instrument, attachment and detachment of the measurement instrument to and from a subject, and other factors during the measurement because the light diffuses nonuniformly due, for example, to the veins, muscles, and bones. Therefore, to perform precise measurement, the measurer requires skill.

The present invention has been made to solve the problems with the related art, and an object of the present invention is to provide an apparatus and a method that readily allow noninvasive lipid measurement with no skill of a measurer.

### Solution to Problem

A lipid measuring apparatus according to the present invention includes an irradiator that radiates light having a predetermined light intensity to a predetermined site of a living body from outside the living body toward inside the living body, a light intensity detector that detects a light arrival range in the living body based on a light intensity of light emitted from the living body, and a controller that calculates a predetermined light arrival range parameter based on the light arrival range and calculates lipid concentration in the living body based on the light arrival range parameter.

A lipid measuring method according to the present invention includes an irradiation step of radiating light having a predetermined light intensity to a predetermined site of a living body from outside the living body toward inside the living body, a light intensity detection step of detecting a light arrival range in the living body based on a light intensity of light emitted from the living body, a parameter calculation step of calculating a predetermined light arrival range parameter based on the light arrival range, and a lipid concentration calculation step of calculating lipid concentration in the living body based on the light arrival range parameter.

A lipid measuring apparatus according to the present invention is a lipid measuring apparatus communicably connected to a user apparatus including an irradiator that radiates light having a predetermined light intensity to a predetermined site of a living body from outside the living body toward inside the living body, a light intensity detector that detects a light arrival range in the living body based on a light intensity of light emitted from the living body, and a communication section that transmits the light arrival range detected by the light intensity detector, the lipid measuring apparatus including a controller that calculates a predetermined light arrival range parameter based on the light arrival range transmitted from the user apparatus and calculates lipid concentration in the living body based on the light arrival range parameter.

### Advantageous Effects of Invention

The lipid measuring apparatus and method according to the present invention readily allow noninvasive lipid measurement with no skill of a measurer.

### BRIEF DESCRIPTION OF DRAWINGS

FIG. 1 shows the configuration of a lipid measuring apparatus according to an embodiment.

FIG. 2 shows that light is scattered by lipid in the blood.

FIG. 3 shows the configuration of a control system of the lipid measuring apparatus according to the embodiment.

FIG. 4 shows a light arrival range having a circular shape.

FIG. 5 shows the light arrival range having a distorted shape.

FIG. 6 is a flowchart of a method for operating the lipid measuring apparatus according to the embodiment.

FIG. 7 shows the configuration of a lipid measuring system according to the embodiment.

FIG. 8 shows the configuration of a control system of a lipid measuring apparatus according to the embodiment.

FIG. 9 shows a result of imaging of the light arrival range.

FIG. 10 shows a result of imaging of the light arrival range.

FIG. 11 shows a result of imaging of the light arrival range in the vicinity of the veins.

FIG. 12A compares a change in lipid concentration and a change in the area of the light arrival range.

FIG. 12B compares the change in lipid concentration and the change in the area of the light arrival range.

FIG. 13A shows the relationship between a minimum light arrival distance and the lipid concentration.

FIG. 13B shows the relationship between the minimum light arrival distance and the lipid concentration.

FIG. 14A shows the relationship between a light arrival volume and the lipid concentration.

FIG. 14B shows the relationship between the light arrival volume and the lipid concentration.

FIG. 15 shows the arrangement of an irradiator and a light intensity detector that differs from the arrangement shown in FIG. 2.

FIG. 16 shows an example of the result of imaging based on the arrangement of the irradiator and the light intensity detector shown in FIG. 15.

FIG. 17A shows results of measurement based on the arrangement of the irradiator and the light intensity detector shown in FIG. 15.

FIG. 17B shows results of the measurement based on the arrangement of the irradiator and the light intensity detector shown in FIG. 15.

### DESCRIPTION OF EMBODIMENT

A lipid measuring apparatus according to an embodiment of the present invention and a method for operating the same will be described below in detail with reference to the drawings.

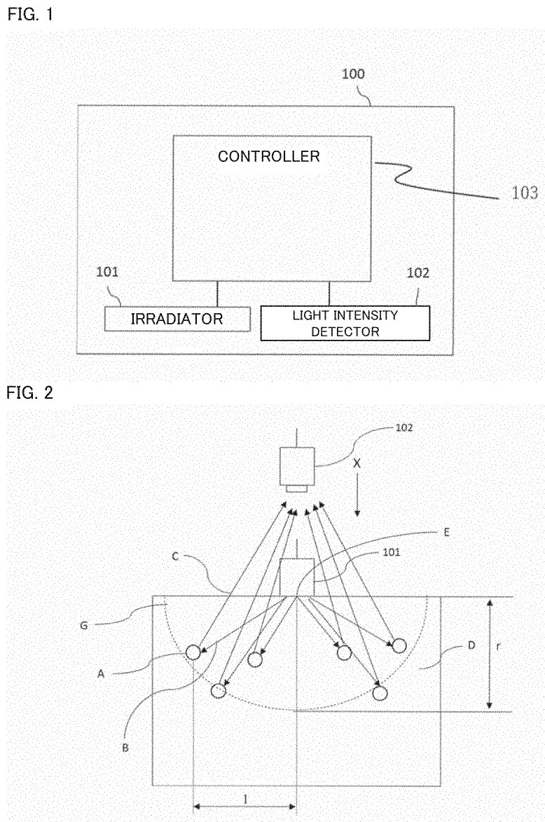

FIG. 1 shows the configuration of the lipid measuring apparatus according to the embodiment.

A lipid measuring apparatus 100 according to the embodiment includes an irradiator 101, which radiates light to a predetermined site of a living body from outside the living body toward the interior of the living body, a light intensity detector 102, which receives light emitted from the living body and detects a light arrival range F in the living body based on the light intensity of the received light, and a controller 103, which calculates a light arrival range parameter based on the light arrival range F detected by the light intensity detector 102 and calculates lipid concentration based on the light arrival range parameter, as shown in FIG. 1.

The irradiator 101 includes a light source for radiating the light to a predetermined irradiation position on the predetermined site of the living body from outside the living body toward the interior of the living body. The irradiator 101 in the embodiment can adjust the wavelength of the radiated light. The irradiator 101 can adjust the range of the wavelength in such a way that the wavelength range does not fall within the range of the wavelengths at which the light is absorbed by inorganic substances of the blood plasma. The irradiator 101 can perform the adjustment in such a way that the wavelength range does not fall within the range of the wavelengths at which the light is absorbed by the cell components of the blood. The cell components of the blood are formed of the red blood cells, white blood cells, and platelets in the blood. The inorganic substances of the blood plasma are formed of water and electrolytes in the blood.

The range of the wavelength of the light radiated by the irradiator 101 is preferably formed of the range shorter than or equal to about 1400 nm and the range from about 1500 to 1860 nm in consideration of the range of the wavelengths at which the light is absorbed by the inorganic substances of the blood plasma. Further, the range of the wavelength of the light radiated by the irradiator 101 is more preferably formed of the range from about 580 to 1400 nm and the range from about 1500 to 1860 nm in consideration of the range of the wavelengths at which the light is absorbed by the cell components of the blood.

The thus set wavelength range used by the irradiator 101 suppresses the influence of the inorganic substances of the blood plasma on the light absorption and the influence of the cell components of the blood on the light absorption of light to be detected by the light intensity detector 102, which will be described later. In the thus set wavelength range, no absorption large enough to identify a substance is present, whereby light energy loss due to the absorption is negligibly small. The light in the blood therefore propagates over a large distance when scattered by lipid in the blood and exits out of the living body.

The irradiator 101 in the embodiment can arbitrarily adjust the time length, for example, for which continuously light or pulsed light is radiated. The irradiator 101 can arbitrarily modulate the intensity or phase of the radiated light.

The irradiator 101 may be formed of a light source having a fixed wavelength. The irradiator 101 may instead be formed of the combination of a plurality of light sources having different wavelengths or the combination of light fluxes having a plurality of wavelengths.

The light intensity detector 102 receives light emitted out of the living body, detects the light intensity of the light, and detects the light arrival range F in the living body.

FIG. 2 shows the light scattered by lipid in the blood. The light (B in FIG. 2) radiated from the irradiator 101 to an irradiation position (E in FIG. 2) on the surface of a living body D arrives at the depth where lipid, such as lipoprotein, is present and is then reflected off in-blood lipid (A in FIG. 2) in the living body D, as shown in FIG. 2. Further, after the radiated light is scattered by the lipid in the blood, and resultant back-scattered light (C in FIG. 2) is emitted from the living body. The light intensity detector 102 detects the light intensity of the back-scattered light C.

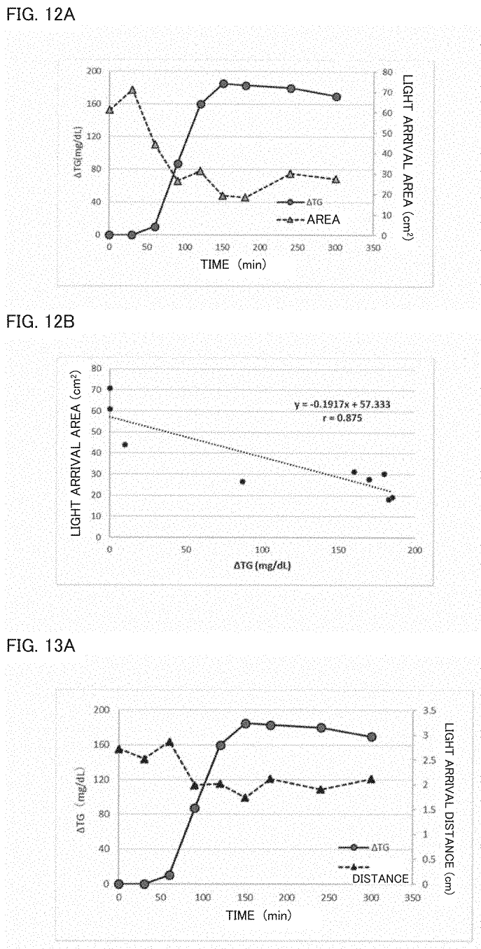

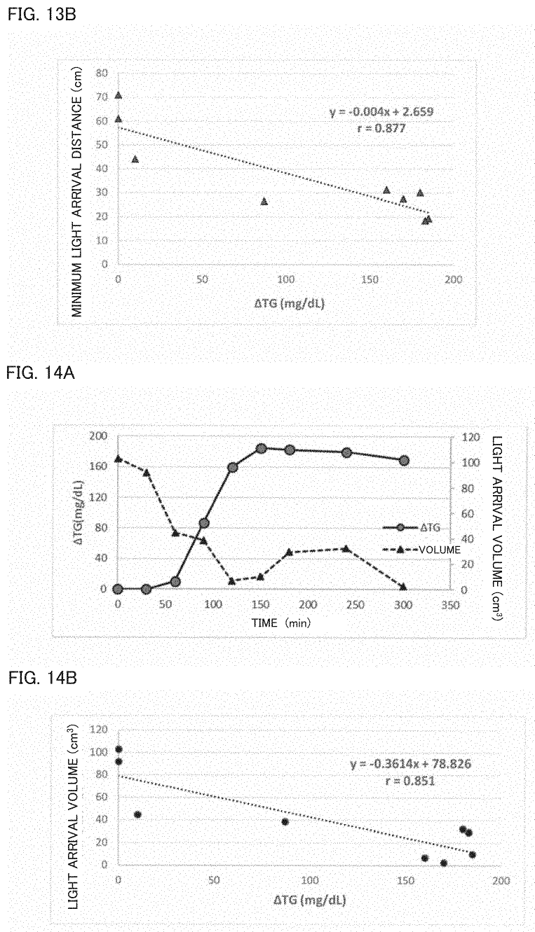

In FIG. 2, the front end of the irradiator 101 is in contact with the living body D and may instead be separate from the living body D by a predetermined distance, as shown in FIG. 13.

The distance from the irradiation position E, to which the irradiator 101 radiates light, to the outer circumference of the range over which the light intensity has a predetermined level (hereinafter referred to as light arrival range F) is called a light arrival distance l, as shown in FIG. 2.

Lipoprotein, which is the target under measurement, has a spherical structure covered with apoprotein and other substances. Lipoprotein is present in the form of a solid-like state in the blood. Lipoprotein is characterized in that it reflects light. In particular, chylomicron (CM), VLDL, and other substances having a large particle diameter and specific gravity contain a large amount of triglyceride (TG) and are characterized in that they are more likely to scatter light. The light intensity detected by the light intensity detector 102 is affected by the light scatted by lipoprotein.

The light intensity detector 102 may be a CCD or CMOS element or any other light receiving element. The light intensity detector 102 may instead be formed of light receiving elements arranged in an array or in a concentric form. To reduce the number of light receiving elements, the light receiving elements may be arranged in the form of a cross or a letter V around the irradiation position E or may be linearly arranged and moved or rotated in the measurement.

In FIG. 2, the light intensity detector 102 is placed immediately above the irradiator 101, but not necessarily, and may be located in any position where the light intensity detector 102 can detect the light arrival range F.

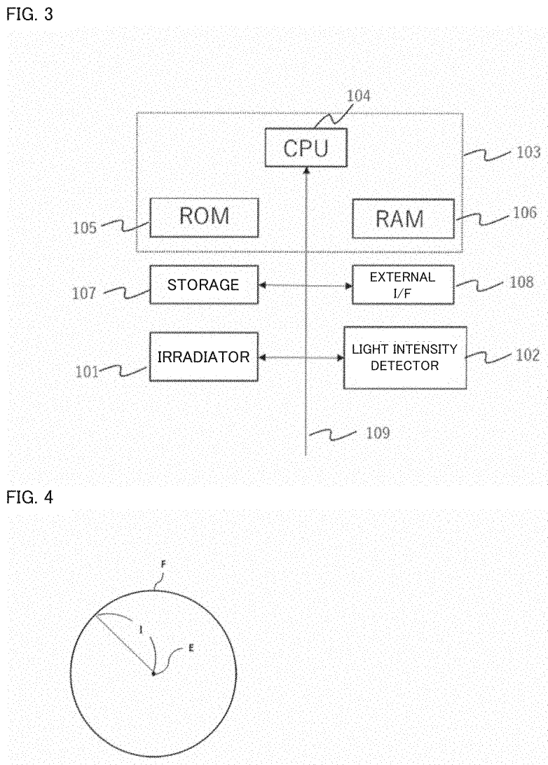

The configuration of a control system of the lipid measuring apparatus 100 will next be described. FIG. 3 is a block diagram of the lipid measuring apparatus 100 according to the embodiment. A CPU (central processing unit) 104, a ROM (read only memory) 105, a RAM (random access memory) 106, a storage 107, an external I/F (interface) 108, the irradiator 102, and the light intensity detector 102 are connected to each other via a system bus 109. The CPU 104, the ROM 105, and the RAM 106 form the controller 103.

The ROM 105 stores in advance a program executed by the CPU 104 and thresholds used by the CPU 104.

The RAM 106 has an area where the program executed by the CPU 104 is developed, a variety of memory areas, such as a work area where the program processes data, and other areas.

The storage 107 stores data prepared in advance on appropriate numerical ranges of static and dynamic parameters. The storage 107 may be an internal memory that stores information in a nonvolatile manner, such as an HDD (hard disk drive), a flash memory, and an SSD (solid-state drive).

The external I/F 108 is an interface for communication with an external apparatus, for example, a client terminal (PC). The external I/F 108 only needs to be an interface that performs data communication with an external apparatus and may, for example, be an instrument (such as USB memory) locally connected to the external apparatus or a network interface for communication via a network.

The controller 103 calculates the light arrival range parameter based on the light arrival range F detected by the light intensity detector 102.

The light arrival range F may be detected by employing a binarization method. The light intensity detected by the light intensity detector 102 is divided into 256 segments from 0 to 255, and the light intensity detector 102 sets a light intensity threshold at 254 so that 255 is taken as the light arrival range F.

The greater the distance between the irradiator 101 and the light intensity detector 102 is, the better the detected light intensity reflects the influence of the scattering. The threshold is therefore not limited to the value described above and may be lowered. In this case, the actual measurement is more likely to be affected by ambient light, and it is therefore preferable to timely set the threshold based on the shape of the apparatus, the degree of light blockage, and the sensitivity of the light receiving element.

In a case where the light receiver is formed, for example, of a PD, an AD value or a voltage value may be used as the threshold, and it is preferable to appropriately set the measurement range used in the measurement based on the intensity of the radiated light, the sensitivity of the light receiving elements, and the degree of light blocking.

In the present example, in a case where the measurement is performed in a darkroom with the ambient light negligible, noise having a magnitude of about 10 in terms of light intensity contaminates a result of the measurement, measurement results having a light intensity of 11 and higher have been examined.

FIG. 4 shows the light arrival range F on the surface of a living body viewed along the direction X in FIG. 2. In the case of capillaries only, the radiated light diffuses in the form of a circle having a radius equal to the light arrival distance l around the irradiation position E, and the light arrival range F has a circular shape on the surface of the living body.

The controller 103 calculates, as the light arrival range parameter, the distance from the irradiation position E in the light arrival range F to the outer circumference (outer edge) of the light arrival range (called light arrival distance l).

The controller 103 further calculates the area of the light arrival range F (called light arrival area S) as the light arrival range parameter. The light arrival area S may instead be calculated from the light arrival distance l. The light arrival area S may still instead be calculated from the number of pixels having the threshold 255. To average measurement errors, the light arrival area S may be calculated in the form of the area of an ellipse having a maximum light arrival distance and a minimum light arrival distance as the major and minor axes.

The controller 103 further calculates the volume of the light arrival range F (called light arrival volume V) as the light arrival range parameter. The light arrival volume V can be calculated by using the following expression: V=(4/3π×a×b×c)/2.

The symbols a, b, and c in the expression are the radii of a sphere that extend in directions x, y, and z of a coordinate system and intersect one another at right angles. In a case where the light arrival range is not distorted, a=b=c is satisfied, whereby l=r in FIG. 2, and the light arrival volume V is (4/3π×l³)/2).

The light arrival range parameter may therefore be any of the light arrival area S, the light arrival distance l, the minimum light arrival distance l2, the light arrival area S and the minimum light arrival distance l2, the ratio or difference between the maximum light arrival distance l1 and the minimum light arrival distance l2, the light arrival volume V, the light arrival volume V and the minimum light arrival distance l2, or the combination thereof.

The controller 103 calculates the lipid concentration in the blood based on the calculated light arrival range parameter (such as light arrival distance l and light arrival area S).

The area over which the radiated light diffuses decreases as the lipid concentration in the blood changes. The reason for this can be inferred as follows: the distance over which the light diffuses decreases as the degree of scattering of the light due to lipid particles in the blood increases. The lipid concentration calculator 104 therefore calculates the lipid concentration in the blood from the light arrival distance l or the light arrival area S. The approach described above does not depend on the measurement site because the measurement can be made, for example, only with information particularly on the capillaries.

The amount of change in lipid concentration and the light arrival area S are so closely related each other that the correlation coefficient is 0.875, as shown in FIG. 12B, whereby the lipid concentration can be calculated from a correlation coefficient specified in advance at least within individual variation.

Instead, the controller 103 may calculate a scattering coefficient from the light arrival range parameter and then calculate the lipid concentration. At a clinical site, the concentration and the turbidity are synonymous with each other in some cases, and the concentration in the present invention includes the turbidity. The controller 103 can therefore use not only the concentration but the number of particles per unit amount, the formazin turbidity, or the scattering coefficient as a result of the calculation.

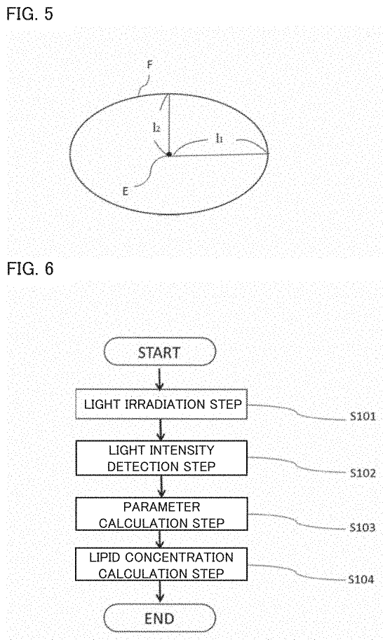

FIG. 5 shows the light arrival range F on the surface of the living body viewed along the direction X in FIG. 2. In a case where the light from the irradiator 101 passes though the veins, the light does not diffuse in the form of concentric circles, and the light arrival range F has a distorted shape having the maximum light arrival distance l1 and the minimum light arrival distance l2 on the surface of the living body. The controller 103 calculates the lipid concentration in the blood from the minimum light arrival distance l2. This approach is an approach that allows the measurement in the case where the light passes through the veins.

The controller 103 may instead calculate the lipid concentration from the light arrival area S and the minimum light arrival distance l2. Information on the veins and capillaries as a whole can therefore be acquired even in a measurement site containing the veins.

The controller 103 may increase the precision of the measurement as information on the veins by calculating the ratio or difference between the maximum light arrival distance l1 and the minimum light arrival distance l2. Further, the controller 103 may instead increase the precision of the measurement as information on the veins by determining the ellipticity of the light arrival range F from the maximum light arrival distance l1 and the minimum light arrival distance l2 or determining the area of the elliptic shape of the light arrival range F.

The lipid measuring apparatus 100 having the configuration described above performs lipid measurement based on a preset program. FIG. 6 is a flowchart of the lipid measurement according to the embodiment.

In an irradiation step (S101), the irradiator 101 radiates continuous light to an irradiation position on a living body.

In a light intensity detection step (S102), the light intensity detector 102 detects the light intensity of the light emitted from the living body around the irradiation position and detects the light arrival range F in the living body based on the light intensity. The light arrival range F detected in the light intensity detection step is sent to a parameter calculation step.

In the parameter calculation step (S103), the controller 103 calculates a predetermined light arrival range parameter based on the light arrival range F. The light arrival range parameter may be the area S of the light arrival range F, the volume V of the light arrival range F, or the distance l from the irradiation position E in light arrival range F to the outer circumference (outer edge) of the light arrival range F. The light arrival range parameter may instead be only the minimum light arrival distance l2, the light arrival area S and the minimum light arrival distance l2, the light arrival volume V and the minimum light arrival distance l2, or the ratio or difference between the maximum light arrival distance l1 and the minimum light arrival distance l2, or the combination thereof. The calculated light arrival range parameter is sent to a lipid concentration calculation step.

In the lipid concentration calculation step (S104), the controller 103 calculates the lipid concentration in the blood based on the light arrival range parameter. In the lipid concentration calculation step, the lipid concentration may be calculated after the scattering coefficient is calculated from the light arrival range parameter.

As described above, the lipid measuring apparatus and method according to the present embodiment readily allow noninvasive lipid measurement with no skill of a measurer by acquiring two-dimensional information on the light intensity of the light emitted from a living body to acquire information on the veins and information on the capillaries.

A lipid measuring apparatus according to another embodiment will next be described. Some portions of the configuration of the lipid measuring apparatus according to the other embodiment are the same as those of the configuration of the lipid measuring apparatus according to the embodiment described above, and different portions will therefore be primarily described.

In the embodiment described above the configuration in which the irradiator 101, the light intensity detector 102, and the controller 103 are integrated with one another has been presented by way of example, but not necessarily. The irradiator 101, the light intensity detector 102, and the controller 103 may be configured as a system in which the irradiator 101 and the light intensity detector 102 are configured as a user apparatus and the controller 103 is provided in a server apparatus connected to the user apparatus.

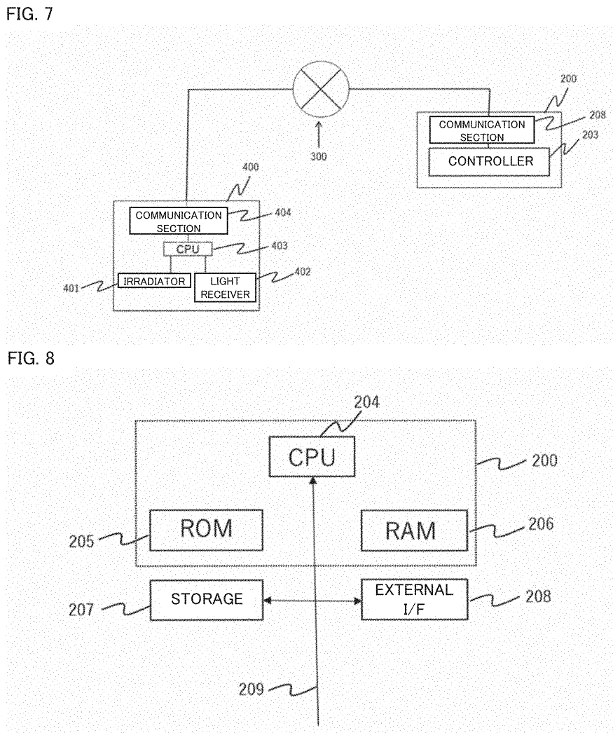

FIG. 7 shows the configuration of a lipid measuring system according to the embodiment. The system includes a lipid measuring apparatus 200, an access point 300, and a user apparatus 400.

The lipid measuring apparatus 200 is an apparatus for calculating lipid concentration by carrying out a predetermined process based on light intensity transmitted from the user apparatus 400. The lipid measuring apparatus 200 is specifically a personal computer or a server apparatus as appropriate depending on the number of apparatuses and the amount of data to be transmitted and received.

The user apparatus 400 is an apparatus possessed by a user and is a standalone apparatus in some cases or is incorporated in a smartphone, a mobile phone, or a wristwatch in other cases. A camera, illumination, a communication function, and the like provided in a smartphone or a mobile phone may be used as an irradiator 401, a light intensity detector 402, and a communication section 404.

The user apparatus 400 includes the irradiator 401, which radiates light, the light intensity detector 402, and the communication section 404. The communication section 404 transmits the light intensity detected by the light intensity detector 402. The functions and actions of the irradiator 401 and the light intensity detector 402 have been described above.

The lipid measuring apparatus 200 includes a communication section 204a and a controller 203. The communication section 204 receives the light intensity transmitted from the communication section 404 via the access point 300 and transmits the light intensity to the controller 203.

The configuration of a control system of the lipid measuring apparatus 200 will next be described. FIG. 8 is a block diagram of the lipid measuring apparatus 200 according to the embodiment. A CPU (central processing unit) 204, a ROM (read only memory) 205, a RAM (random access memory) 206, a storage 207, a communication section (external I/F (interface)) 208 are connected to each other via a system bus 209. The CPU 204, the ROM 205, and the RAM 206 form the controller 203.

The ROM 205 stores in advance a program executed by the CPU 204 and thresholds used by the CPU 204.

The RAM 206 has an area where the program executed by the CPU 204 is developed, a variety of memory areas, such as a work area where the program processes data, and other areas.

The storage 207 stores data prepared in advance on appropriate numerical ranges of static and dynamic parameters. The storage 207 may be an internal memory that stores information in a nonvolatile manner, such as an HDD (hard disk drive), a flash memory, and an SSD (solid-state drive).

The communication section (external I/F) 208 is an interface for communication with an external apparatus, for example, a client terminal (PC). The external I/F 208 only needs to be an interface that performs data communication with an external apparatus and may, for example, be an instrument (such as USB memory) locally connected to the external apparatus or a network interface for communication via a network. The functions and actions of the controller 203 have been described above.

In the embodiment, the light intensity is transmitted from the user apparatus 400 to the lipid measuring apparatus 200 via the access point 300, but not necessarily, and the user apparatus 400 and the lipid measuring apparatus 200 may be directly connected to each other via no access point, and the user apparatus 400 may transmit the light intensity over wired communication, wireless communication, or any other means.

### EXAMPLE

An example of the present invention will be described below, but the present invention is not limited to Example described below.

A lipid measuring apparatus according to the present example readily allows noninvasive lipid measurement with no skill of a measurer by acquiring two-dimensional information on the light intensity of radiated light reflected off and scattered by in-blood lipid in a living body and emitted from the living body to acquire information on the veins and information on the capillaries.

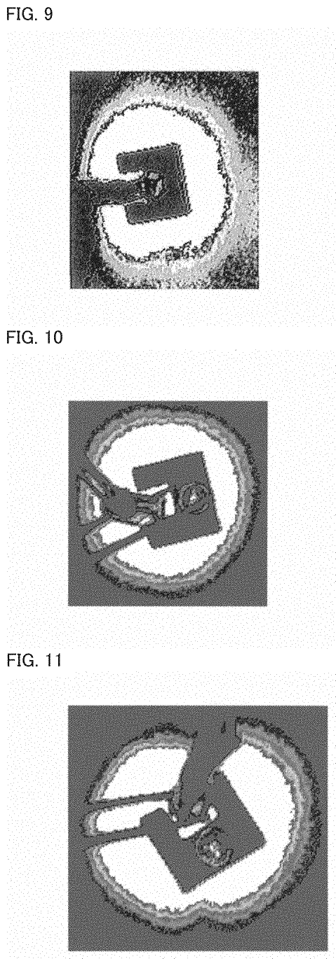

FIG. 9 shows a result of direct radiation of the light from an LED (irradiator 101) onto the skin of a living body and imaging of the light arrival range with an infrared light camera (light intensity detector 102). FIG. 9 shows that the light radiated from the LED (irradiator 101) diffuses in the living body in the form of concentric circles.

FIG. 10 shows a result of the measurement at the same site of the skin of the living body after lipid loading (after blood turbidity increases).

In FIG. 10, the light radiated from the LED (irradiator 101) diffuses in the living body in the form of concentric circles, as in FIG. 9, and comparison between FIGS. 9 and 10 shows that the amount of spread of the light toward the periphery decreases in FIG. 10 as compared with FIG. 9. The data shown in FIG. 10 is data on a measured portion where the veins are invisible in visual inspection.

FIG. 11 shows a result of the measurement in the vicinity of the veins in the forearm. In the veins, a phenomenon considered as attenuation of the light due to the blood is observed, and distorted diffusion instead of diffusion in the form of concentric circles can be observed.

The thus obtained information allows calculation of the lipid concentration by using the following approaches:

(1) Approach to calculation of the lipid concentration from the light arrival area S over which the light diffuses (Approach 1)

(2) Approach to calculation of the lipid concentration from the distortion of the light arrival range F over which the light diffuses due to the veins (Approach 2)

(3) Approach to calculation of the lipid concentration from the light arrival area S over which the light diffuses (Approach 3)

A method for calculating the lipid concentration based on each of the approaches described above will be described below.

(1) Approach to calculation of the lipid concentration from the light arrival area S over which the light diffuses (Approach 1)

In this approach, analysis of portions other than the portions above the veins allows the measurement only with information, for example, on the capillaries, and measurement that does not depend on the measurement site can be made. In a simplified form, the analysis may be made by using the light arrival distance in place of the light arrival range or the light arrival area.

FIG. 12 shows comparison between variation in the lipid concentration and the light arrival area S in a lipid loading test. FIG. 12A shows graphs of the amount of temporal change in TG and the temporal change in light arrival area when lipid is loaded. FIG. 12B shows the correlation between the amount of change in TG and the light arrival area. As shown in FIG. 12A, a decrease in the light arrival area S with an increase in the lipid concentration is observed. The reason for this can be inferred as follows: the distance over which the light diffuses decreases as the amount of scattering due to the lipid particles increases. FIG. 12B shows that the amount of change in TG and the light arrival area are correlated to each other by a degree of correlation of 0.875.

(2) Approach to calculation of the lipid concentration from the distortion of the light arrival range F over which the light diffuses due to the veins (Approach 2)

In the case where the measurement is made through the veins, the light does not concentrically diffuse, and the light arrival range F has a distorted shape. In Approach 2, the maximum light arrival distance l1 and the minimum light arrival distance l2 between the light incident point and the light arrival point are compared.

FIG. 13 shows the relationship between the minimum light arrival distance l2 and the lipid concentration. FIG. 13A shows graphs of shows graphs of the amount of temporal change in TG and the temporal change in the minimum light arrival distance l2 when lipid is loaded. FIG. 13B shows the correlation between the amount of change in TG and the minimum light arrival distance l2 in FIG. 13A. As shown in FIG. 13A, a decrease in the minimum light arrival distance l2 with an increase in the lipid concentration is observed. The reason for this can be inferred as follows: the distance over which the light diffuses decreases as the amount of scattering due to the lipid particles increases. FIG. 13B shows that the amount of change in TG and the minimum light arrival distance l2 are correlated to each other by a degree of correlation of 0.877.

(3) Approach to calculation of the lipid concentration from the light arrival volume V over which the light diffuses (Approach 3)

In this approach, the measurement can be made only with information particularly on the capillaries, and measurement that does not depend on the measurement site can be made.

FIG. 14 shows comparison between variation in the lipid concentration and the light arrival volume V in a lipid loading test. FIG. 14A shows graphs of the amount of temporal change in TG and the temporal change in light arrival area when lipid is loaded. FIG. 14B shows the correlation between the amount of change in TG and the light arrival area in FIG. 14A. As shown in FIG. 14A, a decrease in the light arrival volume V with an increase in the lipid concentration is observed. The reason for this can be inferred as follows: the distance over which the light diffuses decreases as the amount of scattering due to the lipid particles increases. FIG. 14B shows that the amount of change in TG and the light arrival volume V are correlated to each other by a degree of correlation of 0.851.

Further, the combination of Approaches 1and 2 allows calculation of the light arrival area S even in a measurement site containing the veins, and information on the veins and capillaries as a whole can be acquired.

In Approach 2, the precision of the measurement as the information on the veins can be increased by calculating the ratio or difference between the maximum light arrival distance l1 and the minimum light arrival distance l2. Further, in Approach 2, the precision of the measurement as the information on the veins can be increased by determining the ellipticity from the maximum light arrival distance l1 and the minimum light arrival distance l2 or by the area of the ellipse.

Further, to increase the accuracy of the information on the veins, the number of points at which the light radiated from the irradiator 101 is incident is increased, and the position of the veins can also be identified by the information from the plurality of points.

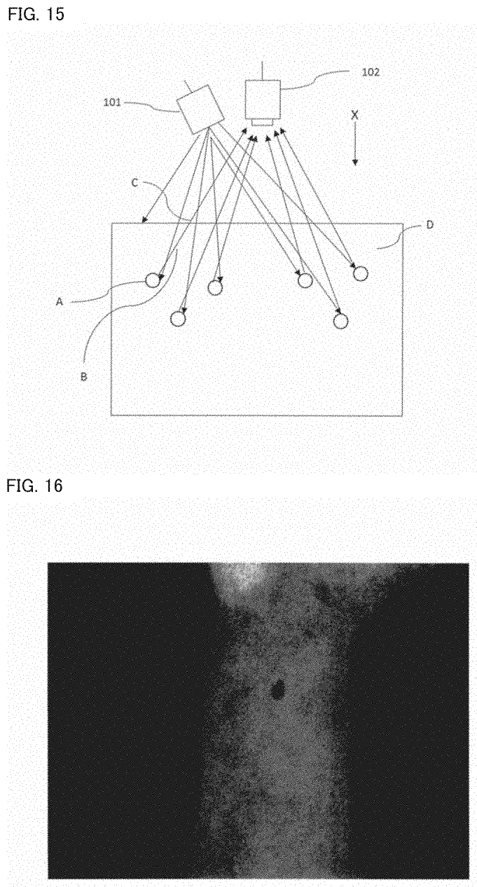

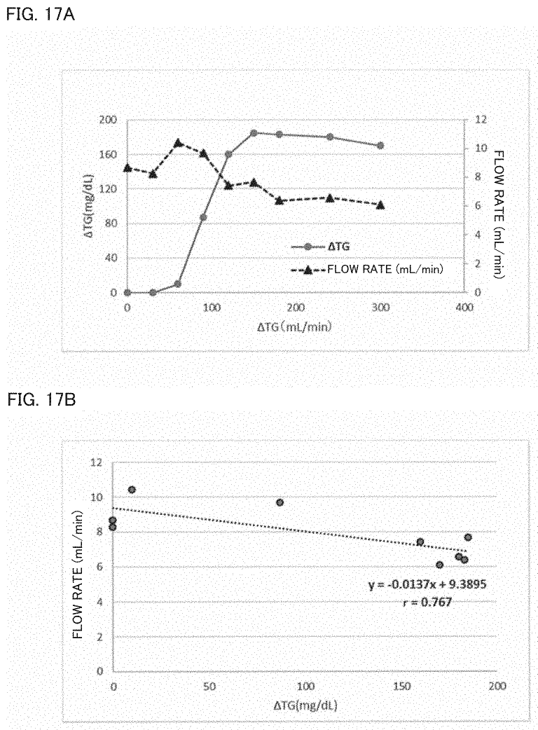

FIG. 15 shows the arrangement of the irradiator 101 and the light intensity detector 102 that differs from the arrangement shown in FIG. 2, and FIG. 16 shows an example of the result of imaging based on the method shown in FIG. 15.

FIG. 16 shows a result of measurement of the blood flow in the capillaries (at light arrival depth of about 1 mm) by using a laser as the irradiator 101, irradiating a wide range with the light from the laser, and measuring speckles produced by the laser light.

The light arrival depth may be adjusted, for example, by adjusting the amount of light from the light source.

FIG. 17 shows a result of the measurement with a subject staying rest and having the same attitude in consideration of the influence of the body temperature, the pulse, and other factors.

FIG. 17A shows graphs of a temporal change in the amount of change in TG and a temporal change in the flow rate when lipid is loaded. FIG. 17B shows the correlation between the amount of change in TG and the flow rate. As shown in FIG. 17A, a decrease in the flow rate with an increase in the lipid concentration is observed. FIG. 17B shows that the amount of change in TG and the flow rate are correlated to each other by a degree of correlation of 0.757. The above result also shows that the lipid concentration can be calculated from information on the blood other than the veins.

Comparison of the information on the veins provided by the present invention with information on the veins provided, for example, by using a method described in Reference Literature allows metabolism information to be obtained more accurately. Comparison between the case where the light source is in contact with a subject and the case where the light source is not in contact with the subject allows information only on the veins to be obtained.

### REFERENCE SIGNS LIST

100: Lipid measuring apparatus
101: Irradiator
102: Light intensity detector
103: Controller

1. A lipid measuring apparatus comprising:
an irradiator that radiates light having a predetermined light intensity to a predetermined site of a living body from outside the living body toward inside the living body;
a light intensity detector that detects a light arrival range in the living body based on a light intensity of light emitted from the living body; and
a controller that calculates a predetermined light arrival range parameter based on the light arrival range and calculates lipid concentration in the living body based on the light arrival range parameter.

2. The lipid measuring apparatus according to claim 1, wherein the light arrival range parameter is based on an area of the light arrival range.

3. The lipid measuring apparatus according to claim 1, wherein the light arrival range parameter is based on a distance from the irradiation position in the light arrival range to an outer circumference of the light arrival range.

4. The lipid measuring apparatus according to claim 1, wherein the light arrival range parameter is based on a volume of the light arrival range.

5. The lipid measuring apparatus according to claim 1, wherein the light arrival range parameter includes a ratio or a difference between a maximum distance and a minimum distance from the irradiation position in the light arrival range to an outer circumference of the light arrival range.

6. The lipid measuring apparatus according to claim 1, wherein the controller calculates a scattering coefficient from the light arrival range parameter and then calculates the lipid concentration.

7. A lipid measuring method comprising:
an irradiation step of radiating light having a predetermined light intensity to a predetermined site of a living body from outside the living body toward inside the living body;
a light intensity detection step of detecting a light arrival range in the living body based on a light intensity of light emitted from the living body;
a parameter calculation step of calculating a predetermined light arrival range parameter based on the light arrival range; and
a lipid concentration calculation step of calculating lipid concentration in the living body based on the light arrival range parameter.

8. The lipid measuring method according to claim 7, wherein the light arrival range parameter is based on an area of the light arrival range.

9. The lipid measuring method according to claim 7, wherein the light arrival range parameter is based on a distance from the irradiation position in the light arrival range to an outer circumference of the light arrival range.

10. The lipid measuring method according to claim 7, wherein the light arrival range parameter is based on a volume of the light arrival range.

11. The lipid measuring method according to claim 7, wherein the light arrival range parameter includes a ratio or a difference between a maximum distance and a minimum distance from the irradiation position in the light arrival range to an outer circumference of the light arrival range.

12. The lipid measuring method according to claim 7, wherein
in the lipid concentration calculation step,
a scattering coefficient is calculated from the light arrival range parameter, and the lipid concentration is then calculated.

13. A lipid measuring apparatus communicably connected to a user apparatus including
an irradiator that radiates light having a predetermined light intensity to a predetermined site of a living body from outside the living body toward inside the living body,
a light intensity detector that detects a light arrival range in the living body based on a light intensity of light emitted from the living body, and
a communication section that transmits the light arrival range detected by the light intensity detector,
wherein the lipid measuring apparatus comprises a controller that calculates a predetermined light arrival range parameter based on the light arrival range transmitted from the user apparatus and calculates lipid concentration in the living body based on the light arrival range parameter.

14. The lipid measuring apparatus according to claim 13, wherein the light arrival range parameter is based on an area of the light arrival range.

15. The lipid measuring apparatus according to claim 13, wherein the light arrival range parameter is based on a distance from the irradiation position in the light arrival range to an outer circumference of the light arrival range.

16. The lipid measuring apparatus according to claim 13, wherein the light arrival range parameter is based on a volume of the light arrival range.

17. The lipid measuring apparatus according to claim 13, wherein the light arrival range parameter includes a ratio or a difference between a maximum distance and a minimum distance from the irradiation position in the light arrival range to an outer circumference of the light arrival range.

18. The lipid measuring apparatus according to claim 13, wherein the controller calculates a scattering coefficient from the light arrival range parameter and then calculates the lipid concentration.

19. The lipid measuring apparatus according to claim 2, wherein the light arrival range parameter is based on a distance from the irradiation position in the light arrival range to an outer circumference of the light arrival range.

20. The lipid measuring apparatus according to claim 2, wherein the light arrival range parameter is based on a volume of the light arrival range.
